(19) United States
(12) Patent Application Publication
(10) Pub. No.: US 2006/0282073 A1
(43) Pub. Date: Dec. 14, 2006

(54) Implant for treating idiopathic scoliosis and a method for using the same

(76) Inventor: Naum Simanovsky, Yehuda (IL)

Correspondence Address: NATH & ASSOCIATES, 112 South West Street, Alexandria, VA, 22314, US

(21) Appl. No.: 10/550,189

(22) PCT Filed: Mar. 31, 2004
(86) PCT No.: PCT/IL04/00296

§ 371 Date: Jul. 13, 2006

(30) Foreign Application Priority Data
Apr. 3, 2003 (IL) 155222

(51) Int. Cl. A61F 2/30

(52) U.S. Cl. 606/61

(57) ABSTRACT

The present invention concerns implant for treating rotational malfunction of the spinal column comprising a linear plate, a set of retractors and clasping means. The present invention further concerns a method for treating Scoliosis by the implantation of said implant

### FIELD OF THE INVENTION

The present invention generally relates to an implant useful for treating rotational malfunction of the spinal column, and especially Idiopathic Scoliosis.

### BACKGROUND OF THE INVENTION

Scoliosis may be defined as deviation of normal spine in all three directions or planes: frontal (coronal), lateral (sagittal) and transversal (axial). In other words, scoliosis is a complex 3D deformation of the trunk, spine and rib cage. Clinically the most prominent feature of this complex deformity is sideward curvature of the trunk accompanied by the hump of the rib. The most common variant of a scoliotic deformity is Idiopathic Scoliosis and particularly it's Adolescent type that may rich up to 3% of the adolescent population. The exact cause of this problem is still unknown, which explains term of Idiopathic for this type of scoliosis. The list of clinical problems associated with scoliosis is far beyond the pure cosmetic complains. It includes distortion of abdominal and chest organs and therefore alteration of their functional capabilities, alteration of normal gait with associated pelvic obliquity and many other functional and social difficulties.

Apart from congenital scoliosis, which is caused by congenital anomalies of spinal structure, for idiopathic type of scoliosis no congenital anomalies of vertebras or rib cage are identified. This may partially explain the fact, that until present time, despite numerous attempts, no animal model of idiopathic scoliosis was made without purposeful alteration of vertebral structure. Therefore, evaluation of the new methods for treatment is complicated and often empirical, mainly based on the personal experience, and believes of the surgeon. In fact, the principles of treatment of scoliosis remain unchanged during last 70 years from the time of publications of Hibbs, Risser and Fergusson. Historically, the treatment began from attempts of manual correction and different types of holding orthotic devices, than the fusion of spinal column in situ was introduced, and than the treatment modalities advanced to acute surgical correction of the deformed spine. Today, the principles of this surgical correction include two basic steps: first, acute correction of spinal deformity during the surgery and insertion of a holding device, and second, solid fusion of vertebral bodies in the position of gained correction, by insertion of bone graft during the same surgical procedure.

The Idiopathic Scoliosis is not acute illness and with time vertebrae becomes secondary deformed. Surgeons who treat a scoliosis know about-deformed shape of scoliotic vertebrae, especially this deformity is prominent on computer tomography evaluation. Apical vertebrae are the most deformed and they appear twisted on the axial CT images.

For correction of scoliotic deformity a different types of holding devices were introduced. All of them are based on acute manual correction of deformity during the surgery and insertion of holding rods or plates engaged to the vertebral body or vertebral prominences by different anchoring devices. The classical example of such approach is the worldwide known Harrington's rods instrumentation. Later, a segmental instrumentation of different types was developed. Debousset and Cotrel have suggested an instrumentation system that is currently considered as a gold standard instrumentation and includes correction in all three planes, but correction is performed acutely and rotational component of correction is limited (See “Instruments in the Treatment of Vertebral Column Deformities”, Orthopade, 1989, 18:118-127). Only recently investigators began to look for dynamic properties of such devices and start with using different types of metalwork with elastic properties, but these new inventions are still based on acutely performed correction and are aimed for preservation of this correction by holding devices till biological fusion of spinal column is achieved. Other efforts are aimed for minimize the number of fused segments of deformed spinal column and thus to increase the movement of free spinal segments.

Methods of treatment Idiopathic Scoliosis known today are characterized by an extensive traumatic nature and considerable morbidity. Furthermore, spinal instrumentation usually consists of massive amount of metal, that remains in the human body for many years and often forever only recently investigators begin to realize the side effects of long-standing metal implants on human body. It is still acknowledged that removal of instrumentation is a traumatic procedure, especially from fused spine with distorted anatomy. Lastly, it is now well acknowledged the harmful side effect provided due to partial dissolving of the metallic parts and compositions, and it's different undesired effects that altogether got name of metalloses.

Unfortunately, a device and method for treating Idiopathic Scoliosis which effectively provided for some preservation of natural spinal movement without decreasing the reinforcing properties of the fixation system is not yet exist. Moreover, such a device additionally characterized by predetermined 3D rotational abilities is substantively needed.

### SUMMARY OF THE INVENTION

It is hence the aim of the present invention to provide an implant useful for treating rotational malfunction of the spinal column wherein said device is adapted to apply pure rotational progressive forces. Said implant comprising a linear plate, a set of connectors and clasping means. The linear plate is having a longitudinal axis adapted to exceed from an apex of the upper scoliotic curve to an apex of the lower scoliotic curve. It is characterized by predetermined lateral dynamic de-rotation properties, having a spring-like means to torque in axial plane. The set of connectors permits free movements of the spine in coronal, longitudinal and/or sagittal directions. The set of anchors are interconnecting said plate with the spinal column, each of said anchor is having a proximal and distal portions. The proximal portion is having means to be reversibly affixed on any position along the longitudinal axis of said plate. The said distal portion is having a connecting means to entrap the spinal column in at least two locations. The clasping means are adapted to clasp the spinous process portion of the spinal column effectively, in the manner the spinal column is to be rotate in a predetermined measure at the time the anchors are entrapping the spinal column and the linear plate is torqued.

By one preferred embodiment each of the anchors comprises anchor parts and a triangular shaped base. The anchor parts comprise a grip in the distal portion of the device adapted to be entrapped into the spinal column; and a base part in the proximal portion of the device.

The triangular shaped base is adapted to interconnect said base part of the anchor with the linear plate. The triangular shaped base is preferably interconnecting the base part of the anchor with the linear plate by a means of an immobilizer, preferably comprising V-shaped or U-shaped clasping means, adapted to clasp the spinous process portion of the spinal column effectively.

Additionally or alternatively, the triangular shaped base comprises a flat distal surface having two triangular or curved protruded grips facing each other; said grips comprising means to immobilize the immobilizer. The space between one grip to the other is such that the width of said space is the width of the linear plate so a predetermined coronal, longitudinal and/or sagital movement of the plate is provided.

The aforementioned anchors are preferably selected from hook-like members; screw-like members, pins, hooks, clasps, fasteners, clips, nails or any combination thereof, or any equivalent construction.

The above-mentioned implant is especially adapted for the correction of Idiopathic Scoliosis. More specifically, said implant is adapted to treat of Idiopathic Scoliosis either exceeded from the thoracis to the lumbar spine or from an apex of any upper scoliotic curve to an apex of the lower scoliotic curve. In addition, said implant may be adapted to treat of Idiopathic Scoliosis comprising more than two apexes. In this case, the implant assembly comprising linear plates in number of said apexes minus one. The number of the sets of anchors is equal the number of the spinal apexes.

It is also in the scope of the present invention wherein the shape of the linear plate is selected from a polygon form, a rod-like form, a sheet-like form, a helical form, a spring, a frame comprising parallel enforcing structures, a bundle of fibers, a screw-like member, a network of warp and weft enforcement, a porosive matrix or any combination thereof. The plate shaped be composed of any bio-compatible material used in orthopedics. Preferably, the linear plate is made of 304 or 316 Stainless Steel, composite materials, shape memory materials or any combination thereof.

The implant according to claim 1, wherein the moment force is tailor made by the physician. The amount of forces that can be produced is depends on the dimensions of the longitudinal plate and can be changed from about 5 lbs/cm to about 150 lbs/cm or more.

The aforementioned implant as defined in any of the above is preferably consisting in at least a portion the anchors as hereto described in FIG. 3 or 4.

It is a second aim of the present invention to provide a useful method for treating rotational malfunction of the spinal column by a means of the implant as defined and described above. Said method comprising the steps as follows: (a) exposing the spinal column over the apex of the proximal (upper) scoliotic curve; (b) placing the anchors to the higher scoliotic curve; (c) placing the anchors to the lower scoliotic curve; (d) making the subcutaneous tunnel between the two operating wounds by blunt dissection under superficial fascia; (e) placing the spring-plate into the subcutaneous tunnel; and (f) twisting the distal (lower) end of the spring-plate along its longitudinal axis in the opposite direction to the proximal (upper) end of the spring-plate. The method is ended by suturing the operative wounds in usual fashion. Most particularly, the aforementioned rotational malfunction of the spinal column to be treated by a means of the method defined in the present invention is Idiopathic Scoliosis.

It is in the scope of the present invention, wherein the step of exposing the spinal column over the apex of the proximal scoliotic curve comprising the following procedure: (a) making straight midline skin incision centered over the apex of the proximal scoliotic curve; (b) deeping the incision to the level of the spinous processes; so the base part of the apical vertebra is extraperiosteally exposed from each side of it; (c) extending the extraperiosteal dissection sideways from the spinous process; and (d) going with dissection and retraction until the middle part of the transverse process on each side of the apical vertebra is exposed.

It is also in the scope of the present invention, wherein the step of placing of the spring-plate into the subcutaneous tunnel comprising the following procedure: (a) inserting the proximal end of the spring-plate into the slot under the connecting plate of the anchors assembly; and (b) securing the spring-plate to the anchors assembly by tightening of the two small screws.

It is also in the scope of the present invention, wherein the step of placing the anchors comprising the following stages: (a) placing the self-retaining retractors adjacent to the spinal column to hold the entire incision open and exposed; (b) placing the hook part of the anchor by sliding the tip of it under the base of the transverse process; (c) performing the same procedure on the other side of the vertebra; (d) fixating the triangular slope-block part to the flat surface of the anchor located on the convex side of the scoliotic curve; (e) pushing the anchors towards the middle line and to each other until they contact above the spinous process of the apical vertebra and intact supraspinous ligament in the manner that no ligament tissue is crushed between their docking parts; and (f) immobilizing both anchors by placing the connecting plate on the upper flat surfaces of the anchors and loosely fixating the connecting plate.

It is acknowledged that according to the aforementioned method as defined above, the step of placing the anchors to the lower scoliotic curve, is comprised of the step of performing a separate incision on the level of the apical vertebra of the distal (lower) scoliotic curve wherein the connecting plate is affixed only to one anchor located on the concave side of the scoliotic curve so the triangular slope-block is located on the opposite side to the triangular slope-block of the upper anchor assembly.

It is also in the scope of the present invention wherein the step of twisting the distal end of the spring-plate along its longitudinal axis in the opposite direction to the proximal (upper) end of the spring-plate comprising the following procedure: (a) adjusting the spring-plate to the flat surfaces of the distal anchor assembly; and (b) fixating the spring plate under the connecting plate using two small screws on each end of the connecting plate.

### BRIEF DESCRIPTION OF THE FIGURES

In order to understand the invention and to see how it may be carried out in practice, a preferred embodiment will now be described, by way of a non-limiting example only, with reference to the accompanying drawings, in which:

FIG. 1A schematically presents a 3D view of a preferred embodiment of the implant according to the present invention and FIG. 1B illustrates the main parts of the implant;

FIG. 2A-2K schematically present a front view of a potion of various types of the linear plates;

FIG. 3 schematically presents a 3D view of the anchors, according to one embodiment of the invention;

FIG. 4 schematically presents a 3D view of the anchors, according to another embodiment of the invention;

FIG. 5 schematically presents a cross section of the spinal column;

FIG. 6 schematically presents a cross section of the spinal column entrapped with the implant according to a preferred mode of the present invention;

FIG. 7A-7B schematically present a top view of the implant entrapped onto the spinal column; and,

FIG. 8A-8C schematically present a top view of the coronal, longitudinal and sagital movement, of the linear plate, respectively.

### DETAILED DESCRIPTION OF THE INVENTION

The following description is provided, along all chapters of the present invention, so as to enable any person skilled in the art to make use of said invention and sets forth the best modes contemplated by the inventor for carrying out this invention. Various modifications, however, will remain apparent to those skilled in the art, since the generic principles of the present invention have been defined specifically to provide to device for treating Idiopathic Scoliosis and a method for using the same.

It is hence in the core of the invention to provide a removable device, generally useful for treating rotational malfunction of the trunk and/or spinal column, and especially for the correction of Idiopathic Scoliosis. This novel device is adapted to apply pure rotational progressive forces on the trunk of mammals and/or their spinal column (hereto denoted for convenience in the unified term ‘spinal column’). The spinal column is hereto divided to thoracic (e.g., the upper portion) and the lumbar (e.g., the lower portion).

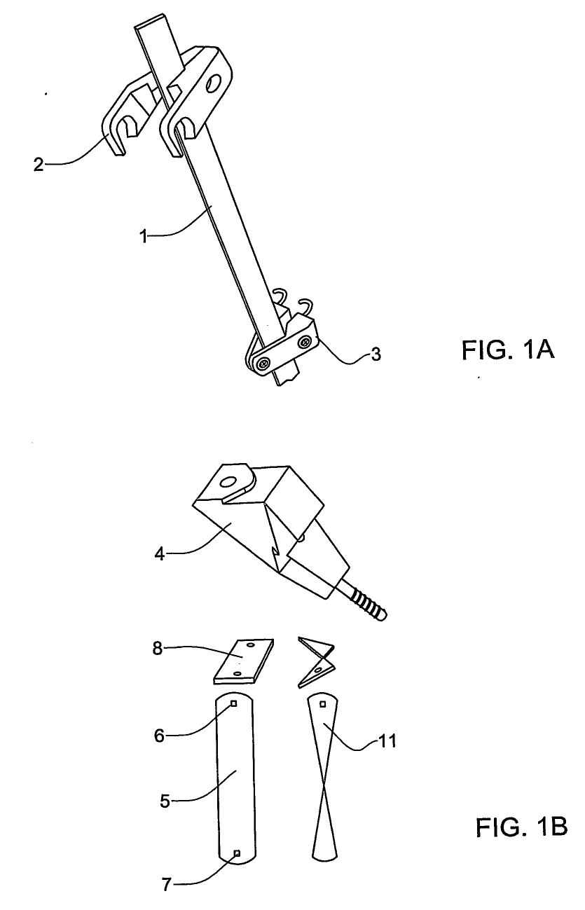

Reference is made now to FIG. 1A presenting a preferred mode of the present invention. The removable implant is comprises of a linear-like enforcer (1) and a set of anchors (2) and (3). The enforcer is thus deforming the trunk by applying pure rotational forces means of the anchors (2 and 3), which are physically held by both the thoracic (anchor 3) and the lumbar (anchor 2) portions of the spinal column. Reference is made now to FIG. 1B presenting a triangular shaped base (4) and the linear plate (5) in its untorqued configuration and its torqued configuration (11). The linear plate (5) preferably comprising at least two stoppers located at the upper and lower portion of the plate (6 and 7, respectively). Those stoppers are adapted to avoid the linear plate (5) to escape from the upper and/or lower connecting immobilizers (8).

The linear-like enforcer (1) is having a longitudinal axis adapted to exceed from the thoracic to the lumbar spine. It is characterized by its lateral dynamic de-rotation properties, adapted to allow the enforcer to coil to a certain axial rotation. The shape of the aforementioned enforcer may be designed in various forms.

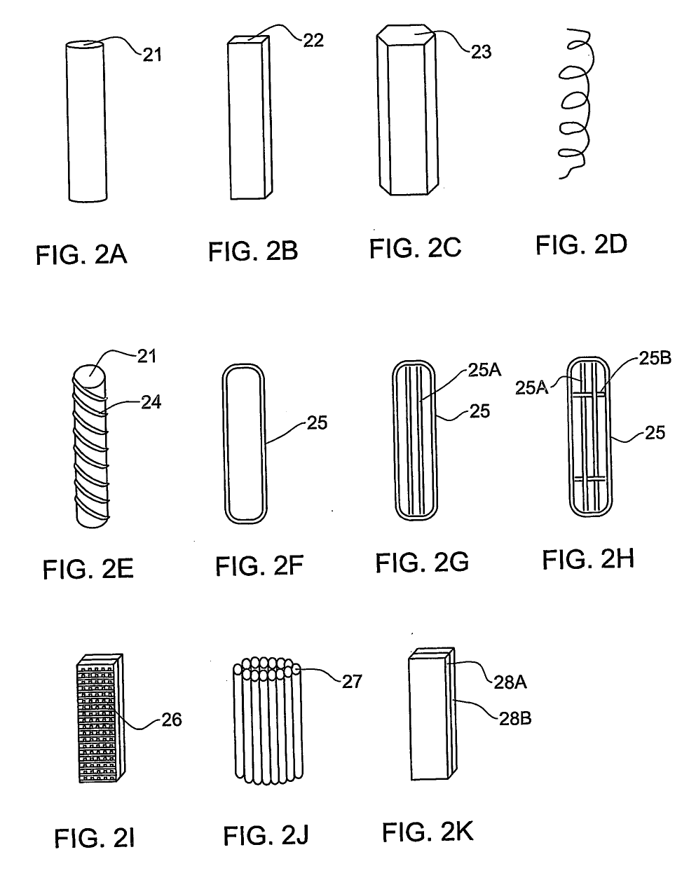

Reference is made now to FIG. 2A to 2K, presenting various forms of the enforcer. FIG. 2A presents a rod-like form having a circular cut (21). FIG. 2B presents a square enforcer, having square cut (22). FIG. 2C presents a polygon form having a polygon cut (23). FIG. 2D presents a helical form (e.g., a spring, a triple helix etc). FIG. 2E presents a male thread screw having a circular cut (21) and helical screw-groves (24). FIG. 2H presents a similar parallel enforcing structure (25 [shown separately in FIG. 2F]) additionally comprising a network of warp and weft enforcement (25A and 25B [shown separately in FIG. 2G]). The mash of said network may varied and differ from case to case. FIG. 2I presents a porosive matrix (26). It is acknowledged in this respect that any combination of the above is possible.

It is further in the scope of the present invention wherein the physician is having accurate means to regulate the torque applied on the spinal column. Hence, reference is now made to FIG. 2J, presenting a schematic view of a bundle of elastic fibers (27). The amount, the type and the size of those fibers generally regulated the force such a bundle can produce. Similarly, reference is made now to FIG. 2K, schematically presenting another preferred embodiment of the present invention, wherein a plurality of linear-like members (28A, 28B etc) are arranged in a stack. By addition or removal of one or more of said members, the physician regulates the desired moment, suitable for the patient at a given time or stage.

It is well in the scope of the present invention wherein the enforcer as defined in any of the above is made of stainless steel, such as 304 or 316 Stainless Steel; and alternatively or additionally, comprising composite materials, shape memory alloys, such as Nitinol shape memory polymeric compositions, or any combination thereof.

It is still in the scope of one preferred embodiment of the present invention wherein the said enforcer dimensions is in the range of about 150 mm×350 mm (length), about 10 mm×30 mm (width), and about 0.5 mm×1.5 mm (thickness). Said enforcer is preferably set to apply a moment of about 5 lbs/cm to 150 lbs/cm. The amount of twisting of the longitudinal plate is in the range of about 40 degrees to 90 degrees depends from the dimension of profile of the triangular connecting block bases.

As said forth above, the aforementioned enforcer of its various types is thus having an accurate and stable deforming means to apply pure rotational forces on the spinal column by a means of a set of anchors, which are physically held by both the thoracis and the lumbors portions of the spinal column. This set of anchors is interconnecting said enforcer with the spinal column. Each of said anchors is having proximal and distal portions. The said proximal portion is having means to be reversibly affixed on any position along the longitudinal axis of said enforcer. The said distal portion is having a hook-like means to entrap the spinal column in at least two locations.

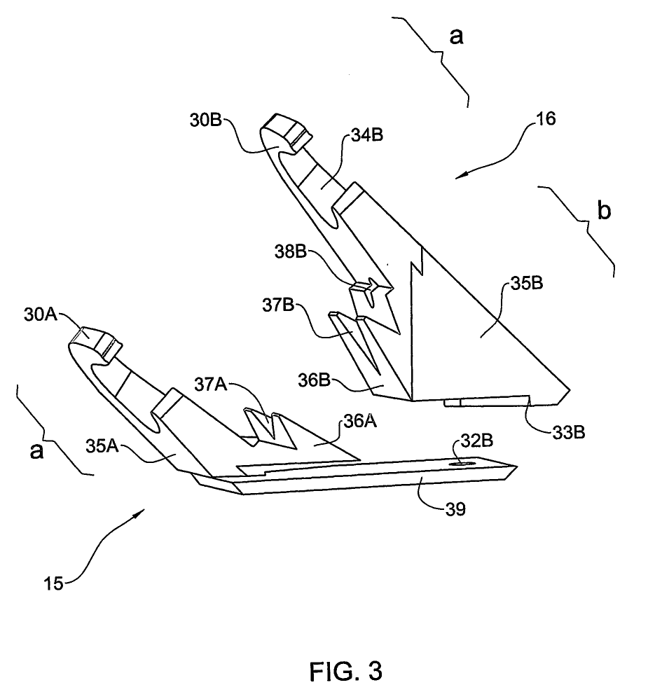

Reference is made now to FIG. 3, presenting one embodiment according to the present invention for a set of anchors adapted to be immobilized or entrapped in the lumbar portion of the spine column. The anchor assembly is comprises of a V-shaped structure having a left anchor (15) and right anchor (16), though assemblies comprising less or more anchors are possible. The anchor assembly is preferably composed of two groups of parts: (a) anchor parts and (b) a triangular shaped base.

The parts of the anchor (a) comprising a grip, e.g., a hook-like member (30A and 30B) in the distal portion of the device; and a base part adapted to clasp the spinal column by a means of a V-shaped members (37B) having a V-shaped recess.

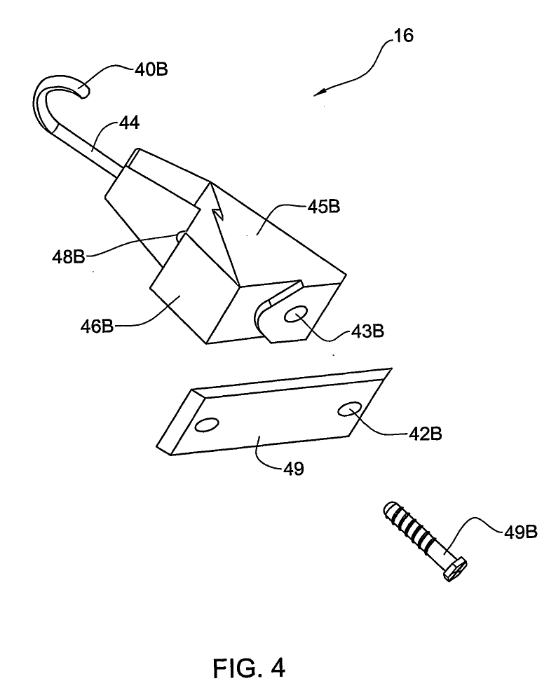

The triangular shaped base comprising at least two bores (38B) and (33B), adapted to connect the base part of the anchor and/or the connecting plate (39), respectively, by means of a fastener, screw, pin or any other connecting means (See 49B in FIG. 4), preferably made of 316 stainless steel.

It is in the scope of the present invention wherein the said anchor parts (a) and the said triangular shaped base (b) are adapted to be interconnected in a plurality of predetermined configurations. Moreover, most of the parts are adapted to be replaced in the manner that the practitioner is capable to ‘tailor’ the most suitable to the specific rotational malfunction of the spinal column.

The hook portion of the anchors (30A and 30B) is extended from the body of the anchors (35A and 35B) by a means of either rotatable or affixed, elastic or non-elastic neck (34), shaped in various possible manners, such as thin neck of circular cut (See 44 in FIG. 4), massive polygonal neck (34) or any combination of the two. Anchor A is assembled with anchor B by means of slides 36A and 36B. Those anchors are further to be fastened by means of reinforcements, adapted to fit V-shaped recess 37A and 37B.

Reference is made now to FIG. 4, presenting one embodiment for a set of anchors adapted to be immobilized or entrapped in the thoracis portion of the spine column. Here, only the right anchor (16) is presented. Said anchor comprising a hook-like member (40B) in the distal portion of the device, and an immobilizer (49) at the proximal portion of the device. The anchor is affixed to the immobilized by a means of a screw, inserted to bore 43A via bore 42B. The massive body (45B) comprises a slide portion (46B), to be assembled with similar slide portion of the second anchor 15, which is not shown here.

It is also in the scope of the present invention to provide useful means to immobilize the anchors into the spinal column. Those means are selected, yet not limited to any of the group of screw-like members, pins, hooks, clasps, fasteners, clips, nails etc. Those terms shall be denoted along the present invention in the short term hereto denoted as a ‘screw’. The screwing means is adapted to be immobilized, entrapped to clap the spinal column in either a reversible or an irreversible manner. Hence, those immobilizing means are adapted to hold the spinal column, to be screwed into the bone etc. It is further acknowledged in this respect that said implant as defined and described in the present invention is adapted to be connected to the pelvic bone. The anchors in this specific case may be designed somewhat different, as so said anchors are screws as defined above.

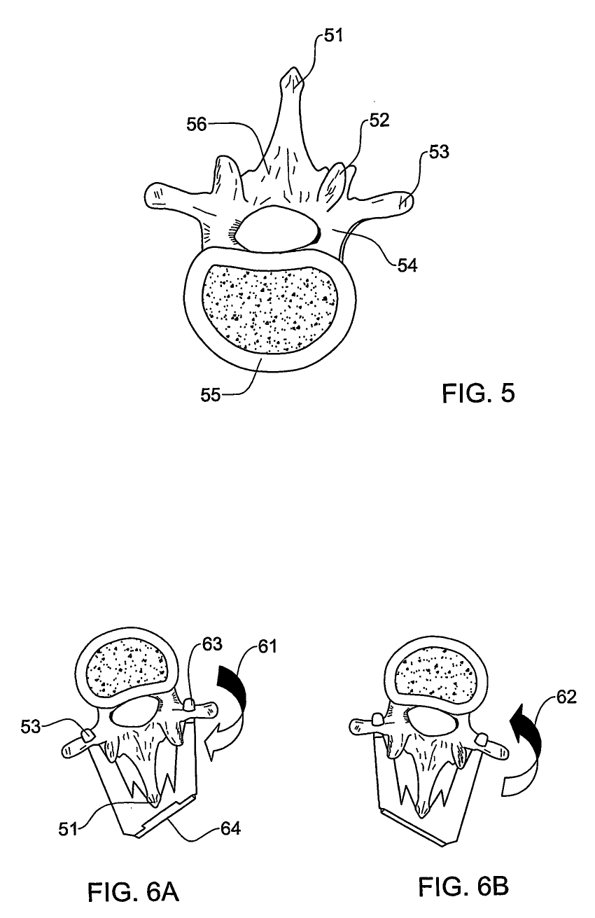

Reference is made now to FIG. 5, presenting a schematic cross section view of the spinal column, comprising spinous process (51); articular process (52); transverse process (53); podicle (54); vertebral body (55) and lamina (56).

Reference is made now to FIG. 6, presenting a schematic cross section view of the spinal column, comprising the implant as defined and described in the present invention. FIG. 6A shows one of the hooks (63) clasping the transverse process (53), while the spinous process (51) is clasps by means of the V-shaped recess (See 37B in FIG. 3). This portion of the implant is in communication with the second portion of the implant, as described in FIG. 6B, by means of the linear plate (64). According to one improvements of the invention, the linear plate (64) is torqued, in the manner the spinal column of the first apex of the scoliotic curve is force to rotate in the direction (61), while the spinal column adjacent to the second apex of the scoliotic curve is forced to rotate to the contrary direction (62).

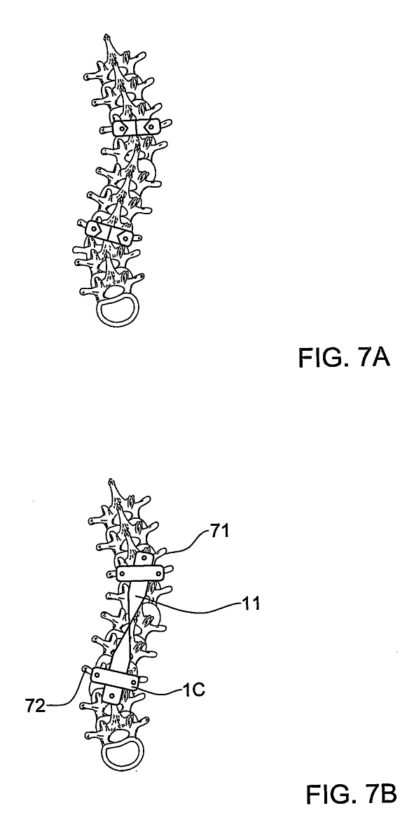

Reference is made now to FIG. 7B, schematically presenting a top view of the spinal column of a patient having a Idiopathic Scoliosis, e.g., having an upper apex (71) and a lower apex (72) of the scoliotic curve, to be rotationally treated by a means of the implant as defined and described in the present invention. The upper hooks are thus located into the spinal column of the apex of the upper scoliotic curve and the lower hooks are located into the spinal column of the apex of the lower scoliotic curve; wherein the linear plate is coiled (11) in the manner the curved spinal column is to be effectively treated. FIG. 7a shows the spinal column from the other side.

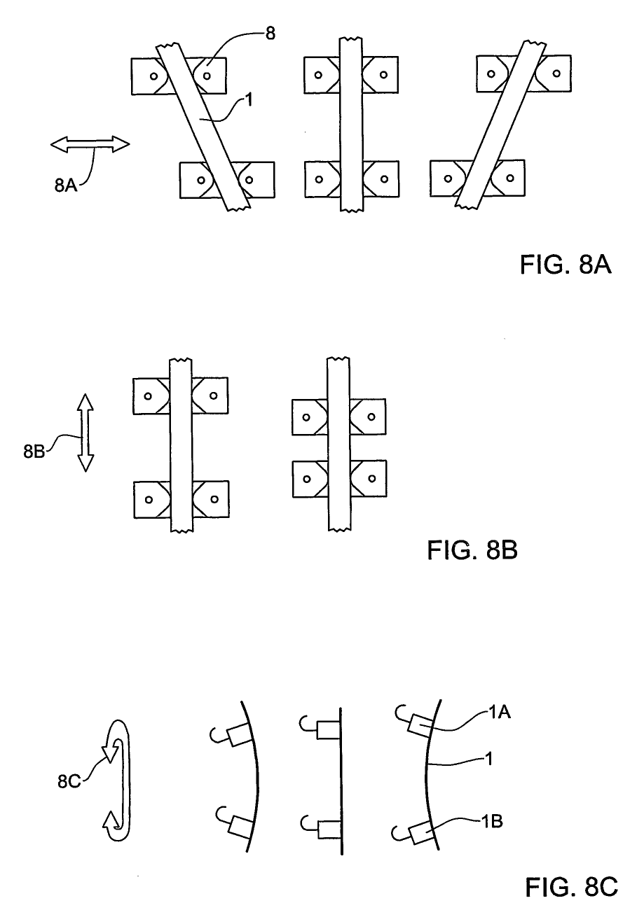

Reference is made now to FIG. 8, presenting a top view of various modes of action of the implant as defined above. Hence, FIG. 8A presents a coronal (side bending) movement in the direction 8A (lateral). FIG. 8B presents a longitudinal movement in the direction 8B (vertical), showing that growth of the spinal column provides no problem for the implant. The escape of the linear plate (1) from the immobilizer (8) is avoided by a means of stoppers (See in 6 and 7 in FIG. 1B). Finally, FIG. 8C presents a sagital (flexion-extension) movement in the direction 8C. It is further acknowledged in this respect that the immobilizer (8) is preferably comprises of a set of shaped protrusions adapted to provide the aforementioned a coronal, longitudinal and/or any predetermined movement of the linear plate. Said protrusions are preferably characterized by a U or a V contour. The gap between the to oppositely directed apexes of said protrusions is about equal to the width of the linear plate, wherein said gap is preferably exceeding about 1 mm the width of the linear plate.

It is another purpose of the present invention to present method for treating rotational malfunction of the spinal column and especially treating Idiopathic Scoliosis by a means of the implant as defined and described in any of the above.

The core of the method is sequence of steps as defined below: (i) exposing the spinal column over the apex of the proximal (upper) scoliotic curve; (ii) placing the anchors to the higher scoliotic curve; (iii) placing the anchors to the lower scoliotic curve; (iv) making the subcutaneous tunnel between the two operating wounds by blunt dissection under superficial fascia; (v) placing the spring-plate into the subcutaneous tunnel; and then (vi) twisting the distal (lower) end of the spring-plate along its longitudinal axis in the opposite direction to the proximal (upper) end of the longitudinal spring-plate.

The whole medical treatment additionally comprising additional various steps, as defined below. First is the step of placing the patient in a prone position. It is acknowledged in this respect that no excessive pressure exists on the abdomen or on the limbs.

Second step is preparing the patient's back to be operate, such as by decontaminating the surface with a surgical soap solution for 5 to 7 minutes and then with antiseptic solution. Preferably, the area of the operative site is then draped and commercial available plastic steri-drape is used to seal off the skin.

Third step is making straight midline skin incision centered over the apex of the proximal (upper) scoliotic curve. The incision length is approximately along about 2 to 3 spinous processes. Then, the incision is her deepening to the level of the spinous processes. The bleeding is controlled with electrocautery.

After those preparations, the aforementioned method is provided. For the sake of explanation, the above mentioned steps are now to be underlined and explained. The base part of the apical vertebra is extaperiosteally exposed from each side of it. The practitioner is preferably suggested to confirm the right location by using of image intensifier.

The extraperiosteal dissection is extended sideways from the spinous process, while keeping the retractors (e.g., Weitlaner retractors) tight at all times. It is preferably suggested to preserve maximum portion of the muscles and ligaments around, until the middle part of the transverse process on each side of the apical vertebra is been exposed. During said exposure the practitioner is provided by means to reduce the damage the branch of segmental vessel located just lateral to each facet.

The self-retaining retractors are now placed deeper to hold the entire incision open and exposed. The hook part of the anchor is placed by sliding the tip of it under the base of the transverse process. The direction of the hook insertion may be either proximal (cranial) or distal (caudal). The same procedure is subsequently performed on the other side of the vertebra. It is acknowledged that in a case that the surgeon decides to use any other than hook-anchor part, for example screw-anchor part its placement is performed using standard technique for a perpendicular screw placement.

The security of anchors seating is now to be checked by means of fixating the triangular slope-block part, (e.g., by using a small screw) to the flat surface of the anchor located on the convex side of the scoliotic curve. The anchors are then pushed towards the middle line and to each other until they contact above the spinous process of the apical vertebra and intact supraspinous ligament. As both anchors are in contact, the practitioner is advised to make sure that no ligamental tissue is crushed between their docking parts. In case of entrapment, the anchors are been replaced with higher ones in such a way that their docking parts meet above the tip of the spinous process and supraspinous ligament.

Now, both anchors are to be immobilized by placing the connecting plate on the upper flat surfaces of the anchors and loosely fixating the connecting plate by two small screws on each end of it.

The same procedure is now provided through the separate incision on the level of the apical vertebra of the distal (lower) scoliotic curve with one exception: the connecting plate should be fixed only to one anchor located on the concave side of the scoliotic curve. Make sure that the triangular slope-block is located on the opposite side to the triangular slope-block of the upper anchor assembly, because in case of the double curve the apical parts of the both curves are rotated in the opposite directions.

The subcutaneous tunnel between the two operating wounds is then provided by a blunt dissection under superficial fascia. The spring-plate is subsequently inserted into the subcutaneous tunnel. The proximal (upper) end of the spring-plate is inserted into the slot under the connecting plate of the anchor assembly and secures the spring-plate to the anchors assembly by tightening of the two small screws. Now the upper part of the spring-plate is secure.

The distal (lower) end of the spring-plate is twisted along its longitudinal axis in the opposite direction to the proximal (upper) end of the spring-plate. The spring-plate is adjusted to the flat surfaces of the distal (lower) anchor assembly. The spring plate is affixed under the connecting plate using two small screws on each end of the connecting plate. Now the whole spring system is assembled.

Lastly, the security of the anchors is to be checked and all the fixation screws are tightened. The practitioner is now suturing the operative wounds in a usual fashion.

In Vivo Study

A. Experimental Design and Methods

This study employs a rabbit model of Adolescent Idiopathic Scoliosis, to characterize the radiographic and morphologic properties of the idiopathic scoliosis.

5 rabbits, six weeks old, are separated into 3 groups.

Young (6 weeks) female New Zealand White rabbits are used to assess the effect of pure rotational forces on the immature spine.

All animals are individually housed and allowed to acclimate to the facility for days prior to experimental use. The animals survive till full adult size and will be maintained in the animal care facility during the post-operative period for routine feeding and exercise before euthanasia. The animal's general activity, appearance, healing of surgical wounds, weight, and appetite is monitored daily.

Group No 1: 1 rabbit. The typical right thoracic left lumbar idiopathic scoliotic curve is be created by placement of spring-plate implant according to the invention. Implant is removed after curve confirmation by x-ray. The natural behavior of the curve is followed after removal of implant.

Group No 2: 1 rabbit. The atypical left thoracic right lumbar idiopathic scoliotic curve is created by placement of spring-plate device. Implant is removed after curve confirmation by x-ray. The natural behavior of the curve is followed after removal of implant.

Group No 3: 3 rabbits. Typical right thoracic left lumbar idiopathic scoliotic curve and opposite curve are created by placement of spring-plate device in different directions. After confirmation of curve by x-ray the spring-plate device is reoriented in opposite direction for curves treatment. After confirmation of curves disappearance by x-ray, the implant is removed and the consistency of improvement is followed after removal of implant.

Assessment of Magnitude of Scoliotic Curve:

The magnitude of the scoliotic curve is assessed by radiographic plain x-ray. The amount of rotational changes of the apical vertebra is evaluated by use of CT-scan. The scoliotic curve progression or improvement during follow-up period is assessed initially at time of surgery and 3 times due growing process and follow-up till achievement by each animal maturity and full adult size.

The anatomical changes of spine are assessed by dissection of the each rabbit after euthanasia

B. Experimental Design

| | Animals | | | |
|---|---|---|---|---|
| | No in | Operative | Follow-up | Follow-up |
| GroupNo | group | Procedure | Procedures | Length |
| 1 | 1 | Application of | Plain films | 4 mo. |
| | | spring-plate | and CT-scan | |
| | | device for right | assessment | |
| | | thoracic left | | |
| | | lumbar curve | | |
| | | creation. Removal | | |
| | | of implant after | | |
| | | curve achievement. | | |
| 2 | 1 | Application of | Plain films | 4 mo |
| | | spring-plate | and CT-scan | |
| | | device for left | assessment | |
| | | thoracic right | | |
| | | lumbar curve | | |
| | | creation. Removal | | |
| | | of implant after | | |
| | | curve achievement. | | |
| 3 | 3 | Application of | Plain films | 6 mo |
| | | spring-plate | and CT-scan | |
| | | device for right | assessment | |
| | | thoracic left | | |
| | | lumbar curve | | |
| | | creation. Change | | |
| | | of spring-plate | | |
| | | orientation after | | |
| | | curve achievement. | | |
| | | Removal of the | | |
| | | device after | | |
| | | curve improvement | | |

| | Agent name, dose, |
|---|---|
| 4. Agent administered | route @ frequency |
| pre-anesthetic agent(s) | Ketamine 35-50 mg/kg IM × 1 |
| | Xylazine 5-10 mg/kg IM × 1 |
| Perioperatively | Rimadyl IM |
| Anesthetic agent(s) | Isofluorane inhalation via |
| | endotracheal tube |
| Intra-operative agent(s) | Buprenorphine 0.02-0.1 mg/kg |
| | SC q 6-12 hrs |
| | Lactaded Ringer's 10-20 |
| | ml/kg/hr IV or SC bolus |
| | Enrofloxacin (Baytril) 5 mg/kg |
| | IM q 12 × 2 doses |
| Pre- and post-operatively | Marcaine 0.5%(3-5 ml) |
| | Injected IM and SC just |
| | prior to surgical closure |
| | to minimize incisional |
| | discomfort |

C. Surgical Procedure(s)—Rabbits:

After adequate anesthesia, the rabbit is placed in the prone position.

Surgical Procedure for Human Beings:

A. Placing the patient in a prone position. Making sure that no excessive pressure exists on the abdomen or on the limbs.
B. Preparing the patient's back with a surgical soap solution for 5-7 minutes and then with antiseptic solution. The area of the operative site is shaved then draped and plastic steri-drape is used to seal off the skin.
C. Making straight midline skin incision centered over the apex of the proximal (upper) scoliotic curve. The incision length is approximately along 2-3 spinous processes. Deeping the incision to the level of the spinous processes. Control bleeding with electrocautery.
D. Exposing the base part of the apical vertebra extraperiosteally from each side of it. Confirm the right location by using of image intensifier. Extending the extraperiosteal dissection sideways from the spinous process keeping the retractors (Weitlaner retractors) tight at all times. Maximally preserve muscles and ligaments around. Keep going with dissection and retraction until the middle part of the transverse process on each side of the apical vertebra is exposed. During exposure try not to damage the branch of segmental vessel located just lateral to each facet.
E. Placing the self-retaining retractors deeper to hold the entire incision open and exposed. Placing the hook part of the anchor by sliding the tip of it under the base of the transverse process. The direction of the hook insertion may be either proximal (cranial) or distal (caudal). Performing the same procedure on the other side of the vertebra In a case when the surgeon decides to use any other than hook-anchor part, for example screw-anchor part its placement is performed using standard technique for pedicular screw placement.
F. Checking the security of anchors seating. Fixating the triangular slope-block part, using a small screw, to the flat surface of the anchor located on the convex side of the scoliotic curve.
G. Pushing the anchors towards the middle line and to each other until they contact above the spinous process of the apical vertebra and intact supraspinous ligament. As both anchors are in contact, make sure that no ligamental tissue is crushed between their docking parts. In case of entrapment, replace the anchors with higher ones in such a way that their docking parts meet above the tip of the spinous process and supraspinous ligament.
H. Immobilizing both anchors by placing the connecting plate on the upper flat surfaces of the anchors and loosely fixating the connecting plate by two small screws on each end of it.
I. Performing exactly the same procedure (C-H) through the separate incision on the level of the apical vertebra of the distal (lower) scoliotic curve with one exception: the connecting plate should be fixed only to one anchor located on the concave side of the scoliotic curve. Make sure that the triangular slope-block is located on the opposite side to the triangular slope-block of the upper anchor assembly, because in case of the double curve the apical parts of the both curves are rotated in the opposite directions.
J. Making the subcutaneous tunnel between the two operating wounds by blunt dissection under superficial fascia.
K. Placing the spring-plate into the subcutaneous tunnel. Insert the proximal (upper) end of the spring-plate into the slot under the connecting plate of the anchors assembly and secure the spring-plate to the anchors assembly by tightening of the two small screws. Now the upper part of the spring-plate is secure.
L. Twisting the distal (lower) end of the spring-plate along its longitudinal axis in the opposite direction to the proximal (upper) end of the spring-plate. Adjusting the spring-plate to the flat surfaces of the distal (lower) anchor assembly. Fixating the spring plate under the connecting plate using two small screws on each end of the connecting plate. Now the whole spring system is assembled.
M. Checking the security of the anchors and tightening of all the fixation screws. Suturing the operative wounds in usual fashion. Be prepared to the tightness and bulging of the edge of the operating wound above the part of the anchor assembly on the convex side of the scoliotic curve. If the skin edge is difficult to close because of the increased volume of the wound content, make release by gentle full thickness undermining of the edge of the operating wound on the tight side.

Thee corneal reflex, heart rate, response to stimuli, and respiration rat are monitored during the operative procedure and post-operative period. Animals are monitored every 15 minutes for the first two hours post-operatively.

During this time period, temperature, heart rate/pulse, respiratory rate, activity level and general appearance including surgical site are monitored. After this two-hour period, animals are checked hourly until 5 p.m. while in the recovery room.

The animals are given an injection of Rimadil (1.5 mg/kg SC 6-12 hrs) for analgesia and Cefamezine (40 mg/kg IM) for antibiotic prophylaxis. Injection of Rimadil is repeated in the evening so as to ensure that post-procedural pain is minimized. Animals are monitored daily once the rabbits are judged to be clinically stable by the animal's general activity, appearance, healing of surgical wounds, weight, and appetite.

The animals survive till full adult size and will be maintained in the animal care facility during the observation period for routine feeding and exercise before euthanasia. Further, the surgery is designed to avoid production of neurological deficit. Animals sustaining neurological deficits will be immediately removed from the study and euthanazed

For euthanasia after achievement of adult size, animals are premedicated with Acepromazine 0.1-0.2 mg/kg SC,

15 minutes prior to euthanasia. Animals are euthanized with Petobarbitol sodium 150 mg/kg IV bolus. Bilateral thoracotomy is performed to ensure adequacy of euthanasia.

Method(s) of Euthanasia:

For euthanasia after achievement by animal adult size, animal will be premedicated with Acepromazine 0.1-0.2 mg/kg SC.

15 minutes prior to euthanasia. Animals are euthanized with Protobarbitol sodium 150 mg/kg IV bolus. Bilateral thoracotomy will be performed to ensure adequacy of euthanasia.

### EXAMPLE 1

### Treatment of Idiopathic Scoliosis by the Implant of the Invention

The animal model of Adolescent Idiopatic Scoliosis was created successfully in accordance with the procedure stipulated above and the scloroitic curve created was moderate.

The implant of the invention was inserted in animals) and the change in the scliosis was assessed as indicated above

The treatment of scoliosis by the implants was successful as determined by x-ray figures (not shown) which demonstrated that the curve was reduced in all animals that survived back to normal indication the concept of role of rotational component of spinal deformity in scoliosis formation is valid.

The concept of effectiveness of continuous derotational forces for treatment of scoliosis was proved. The effectiveness of designed device was proved

1. An implant useful for treating rotational malfunction of the spinal column wherein said device is adapted to apply pure rotational progressive forces, comprising;
a. a linear plate having a longitudinal axis adapted to exceed from an apex of the upper scoliotic curve to an apex of the lower scoliotic curve, having predetermined axial dynamic de-rotational properties, having a spring-like means to torque in axial plate and permitting free movements in coronal, longitudinal and/or sagital directions;
b. at least two anchors interconnecting said plate with the spinal column, each of said anchors is having a proximal and distal portions;
said proximal portion is having means to be reversibly affixed on any position along the longitudinal axis of said plate;
said distal portion is having a connecting means to entrap the spinal column in at least two locations; and
c. clasping means, adapted to effectively clasp the spinous process portion of the spinal column in the manner the spinal column is to be rotate in a predetermined measure at the time the anchors are entrapping the spinal column and the linear plate is torqued.

2. The implant according to claim 1, wherein each of the anchors comprises;
a. anchor parts, comprising;
i. a grip in the distal portion of the device adapted to be entrapped into the spinal column;
ii. base part in the proximal portion of the device; and
b. a triangular shaped base, adapted to interconnect said base part of the anchor with the linear spring plate and permits holding the linear plate in twisted position.

3. The implant according to claim 2, wherein the triangular shaped base is interconnecting the base part of the anchor with the linear plate by a means of an immobilizer.

4. The implant according to claim 2, wherein the triangular shaped block base comprising V-shaped or U-shaped clasping means, adapted to clasp the spinous process portion of the spinal column effectively.

5. The implant according to claim 2, wherein the triangular shaped base comprising a flat distal surface, comprising;
a. two triangular or curved protruded grips facing each other; said grips comprising means to immobilize the immobilizer; and
b. a space between one grip to the other, wherein the width of said space is about 1 mm more than the width of the linear plate so a predetermined coronal, longitudinal and/or sagital movement of the plate is provided.

6. The implant according to claim 1, wherein the anchors are selected from hook-like members; screw-like members, pins, hooks, clasps, fasteners, clips, nails and any combination thereof.

7. The implant according to claim 1, adapted for the correction of Idiopathic Scoliosis.

8. The implant according to claim 7, adapted to treat of Idiopathic Scoliosis either exceeded from the thoracic to the lumbar or from an apex of the upper scoliotic curve to an apex of the lower scoliotic curve.

9. The implant according to claim 7, adapted to treat of Idiopathic Scoliosis comprising more than two apexes of the scoliotic curve the implant comprising
